(19) United States
(12) Patent Application Publication
(10) Pub. No.: US 2016/0375090 A1
(43) Pub. Date: Dec. 29, 2016

(54) METHOD AND COMPOSITION FOR SYNCHRONIZING TIME OF INSEMINATION IN GILTS

(71) Applicant: JBS UNITED ANIMAL HEALTH II LLC, Sheridan (IN)

(72) Inventors: Stephen Kent WEBEL, Baylis, IL (US); Mark E. SWANSON, Princeton Junction, NJ (US); Michael E. JOHNSTON, Noblesville, IN (US)

(73) Assignee: JBS United Animal Health II LLC, Sheridan, IN (US)

(21) Appl. No.: 15/039,728

(22) PCT Filed: Nov. 26, 2014
(86) PCT No.: PCT/US2014/067542

§ 371 Date: May 26, 2016

Related U.S. Application Data
(60) Provisional application No. 61/909,749, filed on Nov. 27, 2013.

(51) Int. Cl. A61K 38/09; A61K 47/38; A61K 9/00; A61K 47/18; A61D 17/00; A61K 47/02; A61K 47/12; A01K 29/00; A61D 19/02; A61K 31/575; A61K 47/14

(52) U.S. Cl. CPC A61K 38/09; A61K 31/575; A61K 47/38; A61K 9/0034; A61K 47/183; A61K 47/14; A61K 47/02; A61K 47/12; A61K 9/0056; A01K 29/005; A61D 19/02; A61D 17/002

(57) ABSTRACT

Methods and compositions for synchronizing the time of insemination in gilts are provided. More particularly, methods and compositions for synchronizing the time of insemination in gilts using a gonadotropin-releasing hormone and a hormone for synchronizing estrus are provided.

### CROSS REFERENCE TO RELATED APPLICATIONS

This application claims priority under 35 U.S.C. §119(e) to U.S. Provisional Application Ser. No. 61/909,749, filed Nov. 27, 2013, the disclosure of which is incorporated herein by reference.

### FIELD OF THE INVENTION

The invention relates to methods and compositions for synchronizing the time of insemination in gilts. More particularly, the invention relates to methods and compositions for synchronizing the time of insemination in gilts using a gonadotropin-releasing hormone and a hormone for synchronizing estrus.

### BACKGROUND OF THE INVENTION

Gonadotropin-releasing hormone is a peptide of 10 amino acids and is also known as luteinizing-hormone releasing hormone (LHRH). Gonadotropin-releasing hormone is produced in the hypothalamus, and is responsible for the release of follicle-stimulating hormone and luteinizing hormone from the pituitary gland. Gonadotropin-releasing hormone is released from neurons in the hypothalmus, and plays a role in the complex regulation of follicle-stimulating hormone and luteinizing hormone release. Follicle-stimulating hormone and luteinizing hormone, in combination, regulate the functioning of the gonads to produce testosterone in the testes and progesterone and estrogen in the ovaries, and regulate the production and maturation of gametes. For example, follicle-stimulating hormone stimulates the growth and recruitment of immature ovarian follicles in the ovary, and luteinizing hormone triggers ovulation.

There are differences in gonadotropin-releasing hormone secretion between females and males. In males, gonadotropin-releasing hormone is secreted in pulses at a constant frequency, but in females the frequency of the pulses varies during the estrus cycle and there is a large surge of gonadotropin-releasing hormone just before ovulation. Gonadotropin-releasing hormone secretion is pulsatile in all vertebrates, and is necessary for correct reproductive function. Thus, gonadotropin-releasing hormone controls a complex process of follicular growth, ovulation, and corpus luteum maintenance in the female, and spermatogenesis in the male.

Gonadotropin-releasing hormone has been isolated and characterized as a decapeptide. Synthetic forms of gonadotropin-releasing hormone are available and modifications of the decapeptide structure of gonadotropin-releasing hormone have led to multiple gonadotropin-releasing hormone analogs that either stimulate or suppress the release of the gonadotropins, such as luteinizing hormone and follicle-stimulating hormone.

It is important to commercial swine production to maximize reproductive efficiency to make swine production more profitable. Labor intensive methods are presently required, such as daily checks for estrus, to increase the probability of success with artificial insemination in swine, such as gilts and sows. Devoting time, manual labor, and materials costs to daily checks for estrus detection is currently necessary because it is difficult to predict the time of estrus (i.e., to predict the best time for insemination) without using methods requiring daily estrus detection. Accordingly, simpler, less labor intensive, but equally effective methods are needed to optimize the success of insemination of swine, including gilts and sows, to reduce the labor costs, costs of materials, and to increase the profitability of swine production.

High sow replacement rates place significant pressures on replacement gilt management to maintain consistent swine production flow. The variation associated with the ovulatory process in gilts is one of the critically important issues related to optimizing reproductive performance. Therefore, effective treatments to more precisely control ovulation are needed so that all gilts in a group may be inseminated without the need for a daily regimen for monitoring estrus.

### SUMMARY OF THE INVENTION

Applicants have discovered effective treatments to more precisely control ovulation in gilts so that the gilts in a group may be inseminated without the need for a daily regimen for monitoring estrus. The methods and compositions described herein are much simpler than daily estrus detection, but are unexpectedly as effective or more effective than a daily regimen for monitoring estrus, in optimizing reproductive performance of gilts. In one embodiment, the methods and compositions described herein increase pregnancy rate as effectively or more effectively than daily estrus detection.

In one embodiment, a method for synchronizing time of insemination in a gilt is provided. The method comprises the steps of 1) administering to the gilt a hormone for synchronizing estrus, 2) administering to the gilt a single dose of a gonadotropin-releasing hormone for synchronizing ovulation, without administration of any other hormone for synchronizing ovulation, wherein the gonadotropin-releasing hormone is administered on the fifth day after the last daily administration of the hormone for synchronizing estrus, 3) inseminating the gilt, without monitoring estrus, on the sixth day after the last daily administration of the hormone for synchronizing estrus, and 4) monitoring estrus on the seventh day after the last daily administration of the hormone for synchronizing estrus, and i) if the gilt is in estrus on the seventh day after the last daily administration of the hormone for synchronizing estrus, inseminating the gilt on the seventh day after the last daily administration of the hormone for synchronizing estrus, or ii) if the gilt is not in estrus on the seventh day after the last daily administration of the hormone for synchronizing estrus, not inseminating the gilt on the seventh day after the last daily administration of the hormone for synchronizing estrus.

The embodiments described in the clauses below, or any combinations thereof, are also contemplated for use in the invention.
1. A method for synchronizing time of insemination in a gilt, the method comprising the steps of:
administering to the gilt a hormone for synchronizing estrus;
administering to the gilt a single dose of a gonadotropin-releasing hormone for synchronizing ovulation, without administration of any other hormone for synchronizing ovulation, wherein the gonadotropin-releasing hormone is administered on the fifth day after the last daily administration of the hormone for synchronizing estrus;
inseminating the gilt, without monitoring estrus, on the sixth day after the last daily administration of the hormone for synchronizing estrus;
monitoring estrus on the seventh day after the last daily administration of the hormone for synchronizing estrus, and
i) if the gilt is in estrus on the seventh day after the last daily administration of the hormone for synchronizing estrus, inseminating the gilt on the seventh day after the last daily administration of the hormone for synchronizing estrus, or ii) if the gilt is not in estrus on the seventh day after the last daily administration of the hormone for synchronizing estrus, not inseminating the gilt on the seventh day after the last daily administration of the hormone for synchronizing estrus.
2. The method according to clause 1 wherein the gonadotropin releasing hormone has the formula

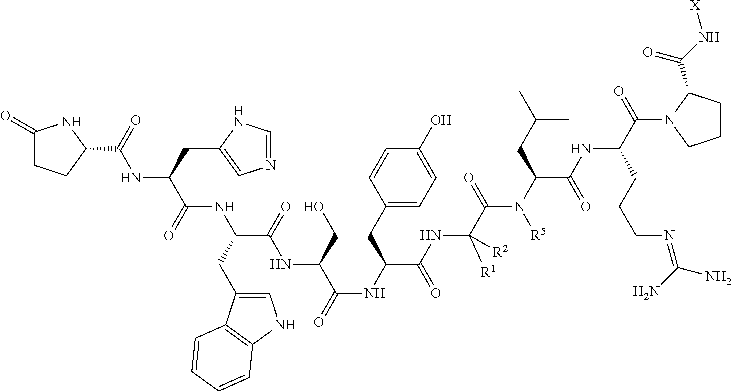

or a solvate, a hydrate, or a pharmaceutically acceptable salt thereof wherein

R¹ and R² are independently in each instance hydrogen, or are independently selected from the group consisting of alkyl, heteroalkyl, cycloalkyl, heterocycloalkyl, haloalkyl, aryl, heteroaryl, arylalkyl, and heteroarylalkyl, each of which is optionally substituted, or R¹ and R² and the attached carbon form a carbocycle or heterocycle;

R⁵ is hydrogen or alkyl; and

X is hydrogen, or X is selected from the group consisting of alkyl, cycloalkyl, heteroalkyl, optionally substituted alkylene-carboxamide, and HNC(O)NR³R⁴, where R³ and R⁴ are in each instance independently selected from the group consisting of hydrogen, alkyl, heteroalkyl and haloalkyl.
3. The method according to clause 2 wherein the gonadotropin-releasing hormone is selected from the group consisting of compounds of the formula of claim 2 wherein
a) R¹ is 1H-indol-3-yl-methyl, R² is hydrogen, X is CH₂(CO)NH₂; R⁵ is hydrogen; and the configuration of the carbon to which R¹ is attached is R;
b) R¹ is hydrogen, R² is hydrogen, X is CH₂(CO)NH₂; and R⁵ is hydrogen;
c) R¹ is 1H-1-benzyl-imidazol-4-yl-methyl, R² is hydrogen, X is ethyl; and R⁵ is hydrogen;
d) R¹ is 2-methylpropyl, R² is hydrogen, X is ethyl; and R⁵ is hydrogen;
e) R¹ is 2-naphthlymethyl, R² is hydrogen, X is CH₂(CO)NH₂; and R⁵ is hydrogen;
f) R¹ is t-butoxymethyl, R² is hydrogen, X is ethyl; R⁵ is hydrogen; and the configuration of the carbon to which R¹ is attached is R;
g) R¹ is benzyl, R² is hydrogen, X is CH₂(CO)NH₂; R⁵ is hydrogen; and the configuration of the carbon to which R¹ is attached is R;
h) R¹ is t-butoxymethyl, R² is hydrogen, X is HN(CO)NH₂; and R⁵ is hydrogen;
i) R¹ is 1H-indol-3-yl-methyl, R² is hydrogen, X is ethyl; and R⁵ is hydrogen;
j) R¹ is methyl, R² is hydrogen, X is hydrogen; R⁵ is hydrogen; and the configuration of the carbon to which R¹ is attached is R;
k) R¹ is 1H-indol-3-yl-methyl, R² is hydrogen, X is ethyl; R⁵ is methyl; and the configuration of the carbon to which R¹ is attached is R;
l) R¹ is methyl, R² is hydrogen, X is CH₂(CO)NH₂; R⁵ is hydrogen; and the configuration of the carbon to which R¹ is attached is R;
m) R¹ is 4-aminobutyl, R² is hydrogen, X is HN(CO)NH₂; R⁵ is hydrogen; and the configuration of the carbon to which R¹ is attached is R;
n) R¹ is methyl, R² is methyl, X is HN(CO)NH₂; and R⁵ is hydrogen; and
o) R¹ is ethyl, R² is hydrogen, X is hydrogen; R⁵ is hydrogen; and the configuration of the carbon to which R¹ is attached is R.
4. The method according to any one of clauses 1 to 3 wherein the insemination is an artificial insemination.
5. The method according to any one of clauses 1 to 4 wherein the gonadotropin-releasing hormone is administered in an effective amount and the effective amount of the gonadotropin-releasing hormone is about 1 μg to about 500 μg.
6. The method according to any one of clauses 1 to 5 wherein the gonadotropin-releasing hormone is administered in an effective amount and the effective amount of the gonadotropin-releasing hormone is about 100 μg to about 300 μg.
7. The method according to any one of clauses 1 to 6 wherein the gonadotropin-releasing hormone is administered in an effective amount and the effective amount of the gonadotropin-releasing hormone is about 200 μg.
8. The method according to any one of clauses 1 to 7 wherein the gonadotropin-releasing hormone is at a concentration of about 50 μg/mL to about 200 μg/mL.
9. The method according to any one of clauses 1 to 8 wherein the gonadotropin-releasing hormone is at a concentration of about 50 μg/mL to about 150 μg/mL.
10. The method of any one of clauses 1 to 9 wherein the gonadotropin-releasing hormone is at a concentration of about 100 μg/mL.
11. The method according to any one of clauses 1 to 10 wherein the dose of the gonadotropin-releasing hormone is administered using a method selected from the group consisting of use of a deposition catheter, manual administration, and injection.
12. The method of clause 11 wherein the gonadotropin-releasing hormone is administered using a deposition catheter.
13. The method of clause 11 wherein the gonadotropin-releasing hormone is administered by injection.
14. The method according to any one of clauses 1 to 13 wherein the hormone is in acetate form.
15. The method according to any one of clauses 1 to 12 or 14 wherein the hormone is administered in a composition comprising a gel.
16. The method according to clause 15 wherein the gel is a polysaccharide selected from the group consisting of celluloses, dextrans, and alginates.
17. The method according to clause 16 wherein the polysaccharide is a cellulose and the cellulose is methylcellulose.
18. The method of clause 17 wherein the gel comprises about 0.5 weight % to about 4.0 weight % of methylcellulose.
19. The method of clause 18 wherein the gel is 1.2% methylcellulose.
20. The method according to clause 15 wherein the gel has a viscosity of about 200 cP to about 5,000 cP.
21. The method of any one of clauses 1 to 12 or 14 to 20 wherein the gonadotropin-releasing hormone is administered intravaginally.
22. The method of clause 21 wherein the gonadotropin-releasing hormone is administered into the anterior vagina.
23. The method according to any one of clauses 1 to 22 wherein the method results in fertility of the gilt.
24. The method according to any one of clauses 2 to 23 wherein in the formula X is H₂CC(O)NH₂, R₁ is hydrogen, and R₂ is

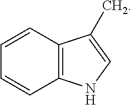

25. The method of any one of clauses 1 to 24 wherein the gonadotropin-releasing hormone is triptorelin.
26. The method of any one of clauses 1 to 25 wherein the hormone that synchronizes estrus is altrenogest.
27. The method according to any one of clauses 1 to 26 wherein the gonadotropin-releasing hormone is in a composition and the composition further comprises a stabilizer wherein the stabilizer is L-methionine.
28. The method of any one of clauses 1 to 27 wherein the gonadotropin-releasing hormone is in a composition with a pH of about 5 to about 6.
29. The method of any one of clauses 1 to 28 wherein the gonadotropin-releasing hormone is in a composition further comprising a preservative.
30. The method of clause 29 wherein the preservative is selected from the group consisting of methylparaben and propylparaben.
31. The method of any one of clauses 1 to 12 or 14 to 30 wherein the gonadotropin-releasing hormone is in a composition and the composition comprises methylparaben, propylparaben, sodium chloride, sodium citrate, L-methionine, citric acid, triptorelin, and methylcellulose.
32. The method of clause 31 wherein the composition comprises methylparaben in an amount of about 0.09% weight per volume, propylparaben in an amount of about 0.01% weight per volume, sodium chloride in an amount of about 0.91% weight per volume, sodium citrate in an amount of about 0.186% weight per volume, L-methionine in an amount of about 0.1% weight per volume, citric acid in an amount of about 0.07% weight per volume, triptorelin in an amount of about 0.01% weight per volume, and methycellulose in an amount that provides a viscosity of about 250 cP to about 400 cP.
33. The method according to any one of clauses 1 to 14 or 21 to 30 wherein the gonadotropin-releasing hormone is in an excipient selected from the group consisting of buffered saline, a liquid alcohol, a glycol, a glucose solution, an ester, an amide, and sterile water.
34. The method of clause 33 wherein the excipient further comprises a pH buffering agent selected from the group consisting of an acetate buffer, a borate buffer, a carbonate buffer, a citrate buffer, a phosphate buffer, hydrochloric acid, sodium hydroxide, magnesium oxide, monopotassium phosphate, bicarbonate, ammonia, carbonic acid, sodium citrate, citric acid, acetic acid, and disodium hydrogen phosphate.
35. The method of any one of clauses 1 to 34 further comprising the step of exposing the gilt to a boar.
36. The method of any one of clauses 1 to 35 wherein the hormone for synchronizing estrus is administered by feeding.
37. The method of any one of clauses 1 to 36 wherein the gonadotropin-releasing hormone is administered about 118 to about 124 hours after the last daily administration of the hormone for synchronizing estrus.
38. The method of any one of clauses 1 to 36 wherein the gonadotropin-releasing hormone is administered about 124 to about 132 hours after the last daily administration of the hormone for synchronizing estrus.
39. The method of any one of clauses 1 to 38 wherein the gilt is inseminated on the sixth day after the last daily administration of the hormone for synchronizing estrus at about 15 to about 32 hours after administration of the gonadotropin-releasing hormone.
40. The method of any one of clauses 1 to 39 wherein the gilt had at least one estrus cycle prior to starting administration of the hormone for synchronizing estrus.
41. The method of clause 40 wherein the at least one estrus cycle occurred 4 to 16 days before starting the administration of the hormone for synchronizing estrus.
42. The method of any one of clauses 1 to 41 wherein the gilt is inseminated on the seventh day after the last daily administration of the hormone for synchronizing estrus.
43. The method of any one of clauses 1 to 41 wherein the gilt is not inseminated on the seventh day after the last daily administration of the hormone for synchronizing estrus.
44. The method of any one of clauses 1 to 43 wherein only one hormone is administered for synchronizing estrus.
45. The method of any one of clauses 1 to 44 wherein the pregnancy rate of the gilt is increased relative to a gilt inseminated based on daily estrus detection.

### DETAILED DESCRIPTION OF THE ILLUSTRATIVE EMBODIMENTS

Applicants have discovered the methods, kits, and compositions described herein that provide for effective treatments to more precisely control ovulation in gilts so that the gilts in a group can be inseminated without the need for a daily regimen for monitoring estrus. The methods and compositions described herein are much simpler than daily estrus detection, but are unexpectedly as effective or more effective than a daily regimen for monitoring estrus, in optimizing reproductive performance of gilts. In one embodiment, the methods and compositions described herein increase pregnancy rate as effectively or more effectively than daily estrus detection.

In one embodiment, a method for synchronizing time of insemination in a gilt is provided. The method comprises the steps of 1) administering to the gilt a hormone for synchronizing estrus, 2) administering to the gilt a single dose of a gonadotropin-releasing hormone for synchronizing ovulation, without administration of any other hormone for synchronizing ovulation, wherein the gonadotropin-releasing hormone is administered on the fifth day after the last daily administration of the hormone for synchronizing estrus, 3) inseminating the gilt, without monitoring estrus, on the sixth day after the last daily administration of the hormone for synchronizing estrus, and 4) monitoring estrus on the seventh day after the last daily administration of the hormone for synchronizing estrus, and i) if the gilt is in estrus on the seventh day after the last daily administration of the hormone for synchronizing estrus, inseminating the gilt on the seventh day after the last daily administration of the hormone for synchronizing estrus, or ii) if the gilt is not in estrus on the seventh day after the last daily administration of the hormone for synchronizing estrus, not inseminating the gilt on the seventh day after the last daily administration of the hormone for synchronizing estrus.

For example, the embodiments described in the clauses below, or any combinations thereof, are contemplated for use in the methods and compositions of the invention.
1. A method for synchronizing time of insemination in a gilt, the method comprising the steps of:
administering to the gilt a hormone for synchronizing estrus;
administering to the gilt a single dose of a gonadotropin-releasing hormone for synchronizing ovulation, without administration of any other hormone for synchronizing ovulation, wherein the gonadotropin-releasing hormone is administered on the fifth day after the last daily administration of the hormone for synchronizing estrus;
inseminating the gilt, without monitoring estrus, on the sixth day after the last daily administration of the hormone for synchronizing estrus;
monitoring estrus on the seventh day after the last daily administration of the hormone for synchronizing estrus, and
i) if the gilt is in estrus on the seventh day after the last daily administration of the hormone for synchronizing estrus, inseminating the gilt on the seventh day after the last daily administration of the hormone for synchronizing estrus, or ii) if the gilt is not in estrus on the seventh day after the last daily administration of the hormone for synchronizing estrus, not inseminating the gilt on the seventh day after the last daily administration of the hormone for synchronizing estrus.
2. The method according to clause 1 wherein the gonadotropin releasing hormone has the formula

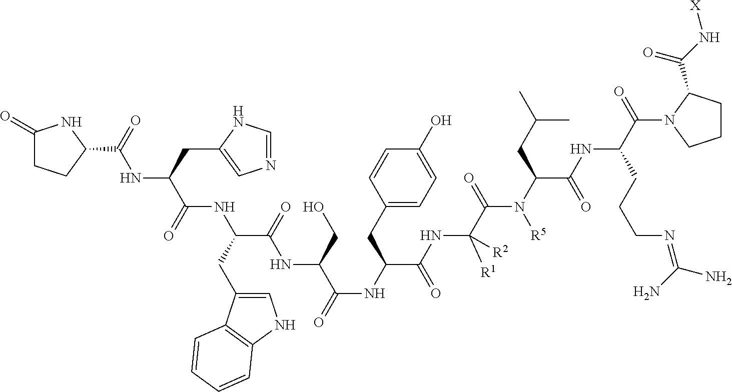

or a solvate, a hydrate, or a pharmaceutically acceptable salt thereof wherein

R¹ and R² are independently in each instance hydrogen, or are independently selected from the group consisting of alkyl, heteroalkyl, cycloalkyl, heterocycloalkyl, haloalkyl, aryl, heteroaryl, arylalkyl, and heteroarylalkyl, each of which is optionally substituted, or R¹ and R² and the attached carbon form a carbocycle or heterocycle;

R⁵ is hydrogen or alkyl; and

X is hydrogen, or X is selected from the group consisting of alkyl, cycloalkyl, heteroalkyl, optionally substituted alkylene-carboxamide, and HNC(O)NR³R⁴, where R³ and R⁴ are in each instance independently selected from the group consisting of hydrogen, alkyl, heteroalkyl and haloalkyl.
3. The method according to clause 2 wherein the gonadotropin-releasing hormone is selected from the group consisting of compounds of the formula of claim 2 wherein
a) R¹ is 1H-indol-3-yl-methyl, R² is hydrogen, X is CH₂(CO)NH₂; R⁵ is hydrogen; and the configuration of the carbon to which R¹ is attached is R;
b) R¹ is hydrogen, R² is hydrogen, X is CH₂(CO)NH₂; and R⁵ is hydrogen;
c) R¹ is 1H-1-benzyl-imidazol-4-yl-methyl, R² is hydrogen, X is ethyl; and R⁵ is hydrogen;
d) R¹ is 2-methylpropyl, R² is hydrogen, X is ethyl; and R⁵ is hydrogen;
e) R¹ is 2-naphthlymethyl, R² is hydrogen, X is CH₂(CO)NH₂; and R⁵ is hydrogen;
f) R¹ is t-butoxymethyl, R² is hydrogen, X is ethyl; R⁵ is hydrogen; and the configuration of the carbon to which R¹ is attached is R;
g) R¹ is benzyl, R² is hydrogen, X is CH₂(CO)NH₂; R⁵ is hydrogen; and the configuration of the carbon to which R¹ is attached is R;
h) R¹ is t-butoxymethyl, R² is hydrogen, X is HN(CO)NH₂; and R⁵ is hydrogen;
i) R¹ is 1H-indol-3-yl-methyl, R² is hydrogen, X is ethyl; and R⁵ is hydrogen;
j) R¹ is methyl, R² is hydrogen, X is hydrogen; R⁵ is hydrogen; and the configuration of the carbon to which R¹ is attached is R;
k) R¹ is 1H-indol-3-yl-methyl, R² is hydrogen, X is ethyl; R⁵ is methyl; and the configuration of the carbon to which R¹ is attached is R;
l) R¹ is methyl, R² is hydrogen, X is CH₂(CO)NH₂; R⁵ is hydrogen; and the configuration of the carbon to which R¹ is attached is R;
m) R¹ is 4-aminobutyl, R² is hydrogen, X is HN(CO)NH₂; R⁵ is hydrogen; and the configuration of the carbon to which R¹ is attached is R;
n) R¹ is methyl, R² is methyl, X is HN(CO)NH₂; and R⁵ is hydrogen; and
o) R¹ is ethyl, R² is hydrogen, X is hydrogen; R⁵ is hydrogen; and the configuration of the carbon to which R¹ is attached is R.
4. The method according to any one of clauses 1 to 3 wherein the insemination is an artificial insemination.
5. The method according to any one of clauses 1 to 4 wherein the gonadotropin-releasing hormone is administered in an effective amount and the effective amount of the gonadotropin-releasing hormone is about 1 μg to about 500 μg.
6. The method according to any one of clauses 1 to 5 wherein the gonadotropin-releasing hormone is administered in an effective amount and the effective amount of the gonadotropin-releasing hormone is about 100 μg to about 300 μg.
7. The method according to any one of clauses 1 to 6 wherein the gonadotropin-releasing hormone is administered in an effective amount and the effective amount of the gonadotropin-releasing hormone is about 200 μg.
8. The method according to any one of clauses 1 to 7 wherein the gonadotropin-releasing hormone is at a concentration of about 50 μg/mL to about 200 μg/mL.
9. The method according to any one of clauses 1 to 8 wherein the gonadotropin-releasing hormone is at a concentration of about 50 μg/mL to about 150 μg/mL.
10. The method of any one of clauses 1 to 9 wherein the gonadotropin-releasing hormone is at a concentration of about 100 μg/mL.
11. The method according to any one of clauses 1 to 10 wherein the dose of the gonadotropin-releasing hormone is administered using a method selected from the group consisting of use of a deposition catheter, manual administration, and injection.
12. The method of clause 11 wherein the gonadotropin-releasing hormone is administered using a deposition catheter.
13. The method of clause 11 wherein the gonadotropin-releasing hormone is administered by injection.
14. The method according to any one of clauses 1 to 13 wherein the hormone is in acetate form.
15. The method according to any one of clauses 1 to 12 or 14 wherein the hormone is administered in a composition comprising a gel.
16. The method according to clause 15 wherein the gel is a polysaccharide selected from the group consisting of celluloses, dextrans, and alginates.
17. The method according to clause 16 wherein the polysaccharide is a cellulose and the cellulose is methylcellulose.
18. The method of clause 17 wherein the gel comprises about 0.5 weight % to about 4.0 weight % of methylcellulose.
19. The method of clause 18 wherein the gel is 1.2% methylcellulose.
20. The method according to clause 15 wherein the gel has a viscosity of about 200 cP to about 5,000 cP.
21. The method of any one of clauses 1 to 12 or 14 to 20 wherein the gonadotropin-releasing hormone is administered intravaginally.
22. The method of clause 21 wherein the gonadotropin-releasing hormone is administered into the anterior vagina.
23. The method according to any one of clauses 1 to 22 wherein the method results in fertility of the gilt.
24. The method according to any one of clauses 2 to 23 wherein in the formula X is H₂CC(O)NH₂, R₁ is hydrogen, and R₂ is

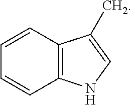

25. The method of any one of clauses 1 to 24 wherein the gonadotropin-releasing hormone is triptorelin.
26. The method of any one of clauses 1 to 25 wherein the hormone that synchronizes estrus is altrenogest.
27. The method according to any one of clauses 1 to 26 wherein the gonadotropin-releasing hormone is in a composition and the composition further comprises a stabilizer wherein the stabilizer is L-methionine.
28. The method of any one of clauses 1 to 27 wherein the gonadotropin-releasing hormone is in a composition with a pH of about 5 to about 6.
29. The method of any one of clauses 1 to 28 wherein the gonadotropin-releasing hormone is in a composition further comprising a preservative.
30. The method of clause 29 wherein the preservative is selected from the group consisting of methylparaben and propylparaben.
31. The method of any one of clauses 1 to 12 or 14 to 30 wherein the gonadotropin-releasing hormone is in a composition and the composition comprises methylparaben, propylparaben, sodium chloride, sodium citrate, L-methionine, citric acid, triptorelin, and methylcellulose.
32. The method of clause 31 wherein the composition comprises methylparaben in an amount of about 0.09% weight per volume, propylparaben in an amount of about 0.01% weight per volume, sodium chloride in an amount of about 0.91% weight per volume, sodium citrate in an amount of about 0.186% weight per volume, L-methionine in an amount of about 0.1% weight per volume, citric acid in an amount of about 0.07% weight per volume, triptorelin in an amount of about 0.01% weight per volume, and methycellulose in an amount that provides a viscosity of about 250 cP to about 400 cP.
33. The method according to any one of clauses 1 to 14 or 21 to 30 wherein the gonadotropin-releasing hormone is in an excipient selected from the group consisting of buffered saline, a liquid alcohol, a glycol, a glucose solution, an ester, an amide, and sterile water.
34. The method of clause 33 wherein the excipient further comprises a pH buffering agent selected from the group consisting of an acetate buffer, a borate buffer, a carbonate buffer, a citrate buffer, a phosphate buffer, hydrochloric acid, sodium hydroxide, magnesium oxide, monopotassium phosphate, bicarbonate, ammonia, carbonic acid, sodium citrate, citric acid, acetic acid, and disodium hydrogen phosphate.
35. The method of any one of clauses 1 to 34 further comprising the step of exposing the gilt to a boar.
36. The method of any one of clauses 1 to 35 wherein the hormone for synchronizing estrus is administered by feeding.
37. The method of any one of clauses 1 to 36 wherein the gonadotropin-releasing hormone is administered about 118 to about 124 hours after the last daily administration of the hormone for synchronizing estrus.
38. The method of any one of clauses 1 to 36 wherein the gonadotropin-releasing hormone is administered about 124 to about 132 hours after the last daily administration of the hormone for synchronizing estrus.
39. The method of any one of clauses 1 to 38 wherein the gilt is inseminated on the sixth day after the last daily administration of the hormone for synchronizing estrus at about 15 to about 32 hours after administration of the gonadotropin-releasing hormone.
40. The method of any one of clauses 1 to 39 wherein the gilt had at least one estrus cycle prior to starting administration of the hormone for synchronizing estrus.
41. The method of clause 40 wherein the at least one estrus cycle occurred 4 to 16 days before starting the administration of the hormone for synchronizing estrus.
42. The method of any one of clauses 1 to 41 wherein the gilt is inseminated on the seventh day after the last daily administration of the hormone for synchronizing estrus.
43. The method of any one of clauses 1 to 41 wherein the gilt is not inseminated on the seventh day after the last daily administration of the hormone for synchronizing estrus.
44. The method of any one of clauses 1 to 43 wherein only one hormone is administered for synchronizing estrus.
45. The method of any one of clauses 1 to 44 wherein the pregnancy rate of the gilt is increased relative to a gilt inseminated based on daily estrus detection.

As used herein, “a gilt inseminated based on daily estrus detection” means the gilt is inseminated based on standard procedures used on farms (i.e., a daily regimen for monitoring estrus) where gilts are monitored for estrus for one to eight or more days to predict the optimal time for insemination.

All of the illustrative embodiments, modifications, and alternative forms described below may be applied to the embodiments described in the preceding paragraphs of this Detailed Description of Illustrative Embodiments section and to the embodiments described in the Summary of Invention.

The methods for synchronizing the time of insemination in gilts described herein include the step of administering to the gilt, a dose of a gonadotropin-releasing hormone. In accordance with one embodiment, the hormone is administered to any porcine species, e.g., sows or gilts (i.e., female pigs prior to first mating), including pubertal gilts, and including gilts that are sexually mature (i.e., have had at least one estrus cycle) or are sexually immature (i.e., have not had an estrus cycle). The methods described herein may result in fertility of the gilt. The methods and compositions described herein are much simpler than daily estrus detection, but are unexpectedly as effective or more effective than a daily regimen for monitoring estrus, in optimizing reproductive performance of gilts. In one embodiment, the methods and compositions described herein increase pregnancy rate as effectively or more effectively than daily estrus detection (i.e., relative to a gilt inseminated based on daily estrus detection).

In one embodiment, a method for synchronizing time of insemination in a gilt is provided. The method comprises the steps of 1) administering to the gilt a hormone for synchronizing estrus, 2) administering to the gilt a single dose of a gonadotropin-releasing hormone for synchronizing ovulation, without administration of any other hormone for synchronizing ovulation, wherein the gonadotropin-releasing hormone is administered on the fifth day after the last daily administration of the hormone for synchronizing estrus, 3) inseminating the gilt, without monitoring estrus, on the sixth day after the last daily administration of the hormone for synchronizing estrus, and 4) monitoring estrus on the seventh day after the last daily administration of the hormone for synchronizing estrus, and i) if the gilt is in estrus on the seventh day after the last daily administration of the hormone for synchronizing estrus, inseminating the gilt on the seventh day after the last daily administration of the hormone for synchronizing estrus, or ii) if the gilt is not in estrus on the seventh day after the last daily administration of the hormone for synchronizing estrus, not inseminating the gilt on the seventh day after the last daily administration of the hormone for synchronizing estrus.

Illustratively, in this embodiment, gilts typically receive a single dose of the gonadotropin-releasing hormone, without administration of any other hormone for synchronizing ovulation, on the fifth day after the last daily administration to the gilt of a hormone for synchronizing estrus (e.g., altrenogest). In this embodiment, the gonadotropin-releasing hormone can be administered, alternatively, on the fourth day after the last daily administration to the gilt of the hormone for synchronizing estrus. In another illustrative embodiment, the gonadotropin-releasing hormone can be administered on the fifth day after the last daily administration to the gilt of the hormone for synchronizing estrus, e.g., about 120 hours after the last daily administration to the gilt of the hormone for synchronizing estrus. As used herein, the phrases “the fourth day after the last daily administration of the hormone for synchronizing estrus”, “the fifth day after the last daily administration of the hormone for synchronizing estrus”, “the sixth day after the last daily administration of the hormone for synchronizing estrus”, and “the seventh day after the last daily administration of the hormone for synchronizing estrus” mean day 4, day 5, day 6, or day 7, respectively, after the last daily administration to the gilt of the hormone for synchronizing estrus, where the last daily administration to the gilt of the hormone for synchronizing estrus is day 0.

In the embodiment described above, the gonadotropin-releasing hormone can be administered at about 110 to about 130, at about 105 to about 136, at about 116 to about 126, about 117 to about 125, about 117 to about 124, about 118 to about 122, about 119 to about 121, or about 120 hours after the last daily administration to the gilt of the hormone for synchronizing estrus. In alternative embodiments the gonadotropin-releasing hormone can be administered at about 117, about 118, about 119, about 120, about 121, about 122, about 123, or about 124 hours after the last daily administration to the gilt of the hormone for synchronizing estrus.

In another embodiment, the gonadotropin-releasing hormone can be administered at about at about 118±2 hr, about 119±2 hr, about 120±2 hr, about 121±2 hr, about 122±2 hr, about 123±2 hr, about 124±2 hr, about 125±2 hr, about 126±2 hr, about 127±2 hr, or about 128±2 hr after the last daily administration to the gilt of the hormone for synchronizing estrus.

In another embodiment, the gonadotropin-releasing hormone can be administered at about 124 to about 134, about 125 to about 133, about 125 to about 132, about 126 to about 130, or about 127 to about 129 hours after the last daily administration to the gilt of the hormone for synchronizing estrus. In alternative embodiments the gonadotropin-releasing hormone can be administered at about 125, about 126, about 127, about 128, about 129, about 130, about 131, or about 132 hours after the last daily administration to the gilt of the hormone for synchronizing estrus.

In yet another embodiment, gilts receiving treatment with the gonadotropin-releasing hormone are typically inseminated without monitoring estrus on the sixth day after the last daily administration of the hormone for synchronizing estrus. The insemination can occur at 15 hours (or 15 hours±2 hr), 16 hours (or 16 hours±2 hr), 17 hours (or 17 hours±2 hr), 18 hours (or 18 hours±2 hr), 19 hours (or 19 hours±2 hr), 20 hours (or 20 hours±2 hr), 21 (or 21 hours±2 hr), 22 hours (or 22 hours±2 hr), 23 hours (or 23 hours±2 hr), 24 hours (or 24 hours±2 hr), 25 hours (or 25 hours±2 hr), 26 hours (or 26 hours±2 hr), 27 hours (or 27 hours±2 hr), 28 hours (or 28 hours±2 hr), 29 hours (or 29 hours±2 hr), or 30 hours (or 30 hours±2 hr) post administration of the gonadotropin-releasing hormone.

In another embodiment, the gilt can be inseminated on the sixth day after the last daily administration of the hormone for synchronizing estrus, without monitoring estrus, for example, about 15 to about 30 hours after administration of the gonadotropin-releasing hormone. In various additional illustrative embodiments, the gilt is inseminated on the sixth day about 17 to about 28 hours after administration of the gonadotropin-releasing hormone, about 18 to about 28 hours, about 20 to about 28 hours, about 22 to about 28 hours, about 22 to about 26 hours, or about 22 to about 24 hours after administration of the gonadotropin-releasing hormone. In the embodiments where the gilt is inseminated on the sixth day after the last daily administration of the hormone for synchronizing estrus, the gilt is inseminated without monitoring estrus. As used herein the phrase “without monitoring estrus” means that tests well known in the art for detecting whether an animal is in estrus are not done.

In yet another embodiment, gilts receiving treatment with the gonadotropin-releasing hormone can be inseminated on the sixth day without monitoring estrus, and then on the seventh day after the last daily administration of the hormone for synchronizing estrus, estrus can be monitored and i) if the gilt is in estrus on the seventh day after the last daily administration of the hormone for synchronizing estrus, the gilt can be inseminated on the seventh day after the last daily administration of the hormone for synchronizing estrus, or ii) if the gilt is not in estrus on the seventh day after the last daily administration of the hormone for synchronizing estrus, the gilt is not inseminated on the seventh day after the last daily administration of the hormone for synchronizing estrus. If estrus is detected on the seventh day after the last daily administration of the hormone for synchronizing estrus, gilts are typically inseminated at 15 hours (or 15 hours±2 hr), 16 hours (or 16 hours±2 hr), 17 hours (or 17 hours±2 hr), 18 hours (or 18 hours±2 hr), 19 hours (or 19 hours±2 hr), 20 hours (or 20 hours±2 hr), 21 (or 21 hours±2 hr), 22 hours (or 22 hours±2 hr), 23 hours (or 23 hours±2 hr), 24 hours (or 24 hours±2 hr), 25 hours (or 25 hours±2 hr), 26 hours (or 26 hours±2 hr), 27 hours (or 27 hours±2 hr), 28 hours (or 28 hours±2 hr), 29 hours (or 29 hours±2 hr), or 30 hours (or 30 hours±2 hr) after the insemination on the sixth day.

In another embodiment, if estrus is detected on the seventh day after the last daily administration of the hormone for synchronizing estrus, the gilt can be inseminated, for example, about 15 to about 30 hours after the insemination on the sixth day. In various additional illustrative embodiments, the gilt is inseminated about 17 to about 28 hours after the insemination on the sixth day, about 18 to about 28 hours, about 20 to about 28 hours, about 22 to about 28 hours, about 22 to about 26 hours, or about 22 to about 24 hours after the insemination on the sixth day. In the embodiments where the gilt is inseminated on the seventh day after the last daily administration of the hormone for synchronizing estrus, the gilt is inseminated if estrus is detected on the seventh day. As used herein, the phrases “monitoring estrus”, “detecting estrus”, or the like mean that tests well known in the art for detecting whether an animal is in estrus are done.

Any of the embodiments described below, are applicable to any of the above-described embodiments. Any of the embodiments described below, including any of the gonadotropin-releasing hormone embodiments and any methods of administration (e.g., injection or intravaginal administration in a gel composition described herein), are also applicable to a method for synchronizing time of insemination in a sow. In one embodiment, the sow is more than four days post-weaning, has recycled, and/or has lost its litter.

Breeding of the gilt may be by any means, including artificial insemination (AI), or through natural breeding. As described, in any embodiment described herein, two breedings (e.g., artificial insemination) may be performed. In yet another embodiment, the gilt is inseminated artificially only one time. In another illustrative embodiment, the methods described herein can further comprise the step of exposing the gilt or the sow to a boar during the process of monitoring estrus to establish the timing of artificial insemination, or, alternatively, not exposing the gilt or the sow to a boar during the process of monitoring estrus to establish the timing of artificial insemination.

In any embodiment described herein, compositions for synchronizing the time of insemination in a gilt comprise: a) a gonadotropin-releasing hormone; and b) a pharmaceutically acceptable pH buffering agent to provide a pH in the range of about pH 4 to about pH 9. The pH of the composition described can range from about 4 to about 9. In other embodiments, the pH can range from about 4 to about 8, from about 4 to about 7, from about 4.5 to about 6.5, about 4.5 to about 6, or from about 5 to about 6.

Further, the gonadotropin-releasing hormone compositions can be produced, in accordance with the dosage form, through a method by appropriately mixing with, diluting with, or dissolving in an additive such as various excipients, disintegrants, binders, salts, lubricants, local anesthetics (e.g., lidocaine), diluents, preservatives, chelating agents, buffers, tonicity agents, antiseptic agents, wetting agents, emulsifiers, dispersants, stabilizers, a solution adjuvant, or combinations thereof.

Illustratively, the compositions comprising the gonadotropin-releasing hormone can be in the form of a gel and the composition can have, for example, a viscosity of about 10 cP (centipoise) to about 300,000 cP. In various illustrative embodiments, the viscosity of the composition can be about 100 cP to about 100,000 cP, about 250 cP to about 400 cP, about 300 cP to about 400 cP, about 500 cP to about 100,000 cP, about 700 cP to about 100,000 cP, about 200 cP to about 20,000 cP, about 200 cP to about 10,000 cP, about 200 cP to about 5,000 cP, about 200 to about 1,000 cP, about 200 cP to about 600 cP, about 100 cP to about 600 cP, about 100 cP to about 500 cP, about 200 cP to about 500 cP, about 200 cP to about 450 cP, or about 100,000 cP to about 250,000 cP. In accordance with various embodiments described herein, the viscosity of the composition can be about 200 cP, about 250 cP, about 300 cP, about 400 cP, about 500 cP, about 1,000 cP, about 15,000 cP, about 20,000 cP, about 30,000 cP, about 40,000 cP, about 50,000 cP, about 75,000 cP, about 100,000 cP, about 200,000 cP, or about 300,000 cP. The viscosity of a solution can be measured using a viscometer, such as a rheometer, based on techniques well-known in the art. The term gel includes viscous solutions that are not solidified.

Typically, the gels as described herein comprise about 0.001 to about 3.0% weight/weight (w/w) of the gonadotropin-releasing hormone or a salt thereof, more typically about 0.5-5.0% (w/w) or about 0.1-5.0% (w/w) of the gonadotropin-releasing hormone or a salt thereof, a preservative, a gel (i.e., a viscosity-modifying agent such as methylcellulose), a buffer to maintain a pH between about 5 to about 6, and a tonicity agent to maintain a tonicity between about 200 to about 400 mOsm/kG.

In accordance with any embodiment described herein, the composition is sufficiently viscous that the composition may adhere to the target tissue (e.g., vaginal tissue) for a sufficient time to deliver an effective amount of the gonadotropin-releasing hormone to the gilt or the sow. The typical viscosity will depend on factors such as, for example, the rate of penetration of the gonadotropin-releasing hormone and the quantity of the gonadotropin-releasing hormone that is applied. Suitable viscosity modulating agents include but are not limited to, ionic and non-ionic water soluble polymers; crosslinked acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof.

The viscosity modulating agent may be in the form of a gel, paste, cream, ointment, and the like. In one embodiment, the composition comprises a gonadotropin-releasing hormone and a gel (e.g., to form a viscous solution), as a viscosity modifying agent, and the gonadotropin-releasing hormone is administered in the composition comprising the gel. In one embodiment, the gel is a hydrogel, a lipogel, or a viscous sol. In another embodiment, the gel is a hydrogel. The gel may be prepared using any method known in the art, for example, such as those methods described in U.S. Pat. Nos. 6,908,623 and 7,456,207, incorporated herein by reference.

In any embodiment described herein, the gel (i.e., a viscosity modifying agent) comprises a polysaccharide. In accordance with the methods and compositions herein described, the polysaccharide may include, for example, alginates and glucose, such as glycogens, starches (e.g., amylose and amylopectin), celluloses, and dextrans. The polysaccharide can be, for example, a methyl, ethyl, or propyl cellulose ester, ether, hydroxy-ether, hydroxy-alkyl, or hydroxy-ester. To achieve the desired viscosity, a sufficient amount of one or more polysaccharides may be used. Typically, about 0.25 to about 10 weight % polysaccharide (based on the total weight of the composition) is desirable. In another embodiment, the weight % of the polysaccharide is about 0.25 weight % to about 3.0 weight %, about 1.0 weight % to about 7 weight %, about 1.0 weight % to about 4.0 weight %, or about 1.0 weight % to about 2.0 weight %. In other embodiments, the weight % of the polysaccharide is about 0.1%, about 0.5%, about 0.75%, about 0.8%, about 0.9%, about 1.0%, about 1.1%, about 1.2%, about 1.4%, about 1.8%, about 2.0%, about 5%, about 8%, or about 10% (all in weight/weight). To increase the viscosity of the composition, the polysaccharide may be used in conjunction with one or more non-polysaccharide viscosifiers known in the art. Examples of non-polysaccharide viscosifiers that can be used in conjunction with one or more polysaccharides include xantham gum, alginic acids and salts thereof, magnesium aluminum silicate, dextrins, sucrose and derivatives thereof, and mixtures thereof. The amount of non-polysaccharide viscosifier, if present, can be about 0.1 weight % to about 10 weight %, depending on the desired viscosity.

In any embodiment described herein, the gel can comprise a cellulose. Illustrative embodiments of the cellulose, as herein described, include methylcellulose, ethylcellulose, hydroxypropyl cellulose, carbomethyl cellulose, hydroxypropyl methyl cellulose, and hydroxyethyl methyl cellulose. The cellulose can be a cellulose derivative, preferably a non-ionic cellulose ester, ether, hydroxy-ether, or hydroxy-ester, or a non-ionic starch derivative. Typically, about 0.25 weight % to about 10 weight % of the cellulose (based on the total weight of the composition) is desirable. In another embodiment, the weight % of the cellulose is about 0.25 weight % to about 3.0 weight %, about 0.5 weight % to about 3.0 weight %, about 0.5 weight % to about 4.0 weight %, about 1.0 weight % to about 7 weight %, about 1.0 weight % to about 4.0 weight %, or about 1.0 weight % to about 2.0 weight %. In other embodiments, the weight % of the cellulose is about 0.1%, about 0.5%, about 0.75%, about 0.8%, about 0.9%, about 1.0%, about 1.1%, about 1.2%, about 1.4%, about 1.8%, about 2.0%, about 5%, about 8%, or about 10% (all in weight/weight). If a uniform gel is desired, dispersing agents such as alcohol, sorbitol, or glycerin can be added, or the gelling agent can be dispersed by tituration, mechanical mixing, or stirring, or combinations thereof.

Acceptable stabilizers for use in the compositions for the methods described herein include, an L-amino acid, such as an L-methionine. In other embodiments, stabilizers that can be used include, but are not limited to, polysaccharides such as acacia, agar, alginic acid, guar gum and tragacanth, gelatin and synthetic and semi-synthetic polymers such as carbomer resins, cellulose ethers, and carboxymethyl chitin. The stabilizer is generally in an amount of about 0.05 to about 10%, about 0.05 to about 5%, about 0.05 to about 2.0%, about 0.05 to about 1.0%, about 0.05 to about 0.5%, about 0.05 to about 0.2%, about 0.1 to about 5%, about 0.1 to about 10%, about 0.1 to about 20%, about 1 to about 5%, about 1 to about 10%, about 1 to about 20% (all in weight/volume). In one embodiment, in the presence of a stabilizer as herein described, the shelf-life of the composition can be at least 12 months, at least 18 months, or at least 24 months. In another embodiment, the composition can be stored at temperatures ranging from about 2° C. to about 8° C. Inert carriers can also be included such as lactose, starch, dextrin, dicalcium phosphate, and calcium sulfate. In one embodiment including a stabilizer, the composition is chemically stable and remains at least 97%, at least 98%, at least 99% pure, at least 99.5% pure, or at least 99.7% pure, for at least three months.

The tonicity agent can be non-ionic or ionic. Illustratively, acceptable tonicity agents for use in the compositions for the methods described herein include, for example, ionic agents such as sodium chloride, potassium chloride, or a balanced salt solution. In accordance with one embodiment, the tonicity agent is present in an amount to achieve a tonicity between about 200-400 mOsm/kG, about 220-380 mOsm/kG, or about 250-340 mOsm/kG. Non-ionic tonicity agents include diols, such as glycerol, mannitol, erythritol, polyethylene glycol, propylene glycol; and sugars such as sucrose and dextrose. The tonicity agent is generally in an amount of about 0.05 to about 10%, about 0.05 to about 5%, about 0.05 to about 2.0%, about 0.05 to about 1.0%, about 0.05 to about 0.5%, about 0.05 to about 0.2%, about 0.1 to about 5%, about 0.1 to about 10%, about 0.1 to about 20%, 0.5 to about 2.0%, about 0.6 to about 2.0%, about 0.5 to about 1.8%, about 0.6 to about 1.8%, about 1.0 to about 5.0%, about 1.0 to about 10%, or about 1.0 to about 20% (all in weight/volume).

In any embodiment described herein, the pH buffering agents for use in the compositions for the methods described herein are those agents known to the skilled artisan to be pH buffering agents or compositions and include, for example, acetate, borate, carbonate, citrate, and phosphate buffers, as well as various biological buffers, for example, TAPS, Bicine, Tris, Tricine, HEPES, TES, MOPS, PIPES, Cacodylate, and MES. Other pH buffering agents include hydrochloric acid, sodium hydroxide, magnesium oxide, monopotassium phosphate, bicarbonate, ammonia, carbonic acid, sodium citrate, citric acid, acetic acid, disodium hydrogen phosphate, borax, boric acid, and the like. The buffering agent is generally in an amount of about 0.01 to about 10%, about 0.02 to about 10%, about 0.02 to about 5%, about 0.02 to about 2.0%, about 0.02 to about 1.0%, about 0.02 to about 0.5%, about 0.05 to about 10.0%, about 0.05 to about 1.0%, about 0.05 to about 0.5%, about 0.05 to about 0.2%, about 0.1 to about 5%, about 0.1 to about 10%, about 0.1 to about 20%, about 1 to about 5%, about 1 to about 10%, about 1 to about 20% (all in weight/volume).

The pH buffering agent used in the formulations described herein can be used at any concentration needed to obtain the desired pH range. For example, the buffering agent can be used at a concentration of about 0.001M to about 1M, about 0.001M to about 2M, about 0.001M to about 5M, about 0.05M to about 0.1M, about 0.05M to about 0.2M, about 0.05M to about 1M, 0.05M to about 2M, about 0.05 to about 5M, about 0.1M to about 1M, about 0.1M to about 2M, about 0.1M to about 5M. Any amount of buffering agent needed to obtain the desired pH range can be used in the formulations described herein. Typically, the pharmaceutically acceptable pH buffering agent can be used to provide a pH in the range of about pH 4 to about pH 9. The pH of the composition herein described can range from about 3 to about 10, or about 4 to about 9. In any embodiment described herein, the pH can range from about 4 to about 8, from about 4 to about 7, from about 4.5 to about 6.5, about 4.5 to about 6, from about 5 to about 6, about 5 to about 5.5, about 4 to about 6, or about 4.5 to about 5.5.

In any embodiment, the composition described herein can comprise one or more pharmaceutically acceptable preservatives. As used herein, the term “preservative” includes an agent or a combination of agents that aids in stabilizing the composition, inhibiting microbial growth, or both. Examples of suitable preservatives include parabens (e.g., methyl, ethyl, propyl, and butyl esters of parahydroxybenzoic acid), propyl gallate, sorbic acid and its sodium and potassium salts, propionic acid and its calcium and sodium salts, “Dioxin” (6-acetoxy-2,4-dimethyl-m-dioxane), “Bronopol” (2-bromo-2-nitropropane-1,3-diol) and salicylanilides such as disbromosalicylanilide, tribromosalicylamilides, “Cinaryl” 100 and 200 or “Dowicil” 100 and 200 (Cis isomer of 1-(3-chloroallyl-3,5,7-triaza-1-azanidadamantane chloride), hexachlorophene, sodium benzoate, citric acid, ethylene diaminetetraacetic acid and its alkali metal and alkaline earth metal salts, butyl hydroxyanisol, butyl hydroxytoluene, phenolic compounds such as chloro- and bromocresols and chloro- and bromo-oxylenols, quaternary ammonium compounds like benzalkonium chloride, aromatic alcohols such as phenylethyl alcohol, benzyl alcohol, etc., chlorobutanol, quinoline derivatives such as iodochlorohydroxyquinolin, and the like. The total amount of preservative, when present, is about 0.005 weight % to about 2 weight %, about 0.001 weight % to 1.0 weight %, about 0.005 weight % to about 0.25 weight %, or about 0.05 weight % to about 0.2 weight %, typically about 0.01 weight % to about 0.1 weight % (all in weight/weight).

In any embodiment, the pharmaceutical composition for the methods described herein can contain a chelating agent, such as those known to those skilled in the art, for example, ethylenediamine tetraacetate (EDTA), diethylenetriaminepentaacetic acid (DTPA), and N,N-bis(carboxymethyl)glycine (NTA), or salts thereof. The composition can contain about 0.003 weight % to about 1.0 weight %, about 0.02 weight % to about 0.2 weight %, about 0.01 weight % to about 1.0 weight %, or about 0.02 weight % to about 0.5 weight % (all in weight/volume) of the chelating agent.

In any embodiment described herein, antimicrobial agents can be included in the compositions for the methods described herein. Such agents may include, but are not limited to 5-chloro-2-(2,4-dichlorophenoxy)-phenol, 8-hydroxyquinoline, copper II compounds, phthalic acid, chlorhexidine, alexidine, hexetidine, sanguinarine, benzalkonium chloride, salicylanilide, domiphen bromide, cetylpyridinium chloride, tetradecylpyridinium chloride, N-tetradecyl-4-ethylpyridinium chloride, octenidine, iodine, sulfonamides, bisbiguanides, phenolics, delmopinol, octapinol, and other piperidino derivatives, and nicin preparations, any suitable antibiotics such as augmentin, amoxicillin, tetracycline, doxycycline, minocycline, metronidazole, neomycin, kanamycin, and clindamycin, and any salts of any of these compounds where applicable, and any combinations of these compounds. In yet another embodiment, anti-fungal compounds can be included, alone or in combination with any of the above-described antimicrobials. Anti-fungal agents that are suitable for use in the compositions described herein include, but are not limited to, nystatin, miconazole, econazole nitrate, clotrimazole, and flucytosine. The antimicrobial or anti-fungal agents can be added to the formulations herein described in an amount of about 0.01 to about 10%, about 0.01 to about 5%, about 0.01 to about 2.0%, about 0.01 to about 1.0%, about 0.01 to about 0.5%, about 0.01 to about 0.2%, 0.05 to about 10%, about 0.05 to about 5%, about 0.05 to about 2.0%, about 0.05 to about 1.0%, about 0.05 to about 0.5%, about 0.05 to about 0.2%, about 0.1 to about 5%, about 0.1 to about 10%, about 0.1 to about 20%, about 1 to about 5%, about 1 to about 10%, about 1 to about 20% (all in weight/volume).

In any embodiment of the compositions for the methods described herein, antioxidants can also be added. For example, antioxidants used herein can include beta-carotene, vitamin E, vitamin C, vitamin A, tocopherol, butylated hydroxytoluene, butylated hydroxyanisole, tertiary-butylhydroquinone, propyl gallate, ascorbic acid, sodium metabisulfite, uric acid, carotenoids, flavonoids, melatonin, and ethoxyquin. The antioxidants can be added to the formulations herein described in an amount of about 0.01 to about 10%, about 0.01 to about 5%, about 0.01 to about 2.0%, about 0.01 to about 1.0%, about 0.01 to about 0.5%, about 0.01 to about 0.2%, 0.05 to about 10%, about 0.05 to about 5%, about 0.05 to about 2.0%, about 0.05 to about 1.0%, about 0.05 to about 0.5%, about 0.05 to about 0.2%, about 0.1 to about 5%, about 0.1 to about 10%, about 0.1 to about 20%, about 1 to about 5%, about 1 to about 10%, about 1 to about 20% (all in weight/volume).

In one embodiment, the gonadotropin-releasing hormone for use in the methods described herein is in a composition and the composition comprises methylparaben, propylparaben, sodium chloride, sodium citrate, L-methionine, citric acid, triptorelin, and methylcellulose. In another aspect, the composition comprises methylparaben in an amount of about 0.09% weight per volume, propylparaben in an amount of about 0.01% weight per volume, sodium chloride in an amount of about 0.91% weight per volume, sodium citrate in an amount of about 0.186% weight per volume, L-methionine in an amount of about 0.1% weight per volume, citric acid in an amount of about 0.07% weight per volume, triptorelin in an amount of about 0.01% weight per volume, and methycellulose in an amount of about 1.2% weight per volume (or with a viscosity of about 250 cP to about 400 cP).

The gonadotropin-releasing hormone composition for the methods described herein contains a gonadotropin-releasing hormone in an amount effective to synchronize the time of insemination in a gilt when used in the methods described herein. As used herein, “gonadotropin-releasing hormone” refers to any gonadotropin releasing hormone, including gonadotropin releasing hormone analogs and derivatives, and gonadotropin releasing hormone agonists and antagonists. In other embodiments, luteinizing hormone or human chorionic gonadotropin, or derivatives or analogs thereof, and combinations thereof can be used in place of, or in combination with the gonadotropin-releasing hormone. As used herein, “luteinizing hormone” refers to any luteinizing hormone, including luteinizing hormone analogs and derivatives, and luteinizing hormone agonists and antagonists. In one embodiment, the luteinizing hormone can be synthetic. In another embodiment, the luteinizing hormone can be LH (see, for example, U.S. Pat. No. 5,444,167, incorporated herein by reference). As used herein, “human chorionic gonadotropin” refers to any human chorionic gonadotropin, including human chorionic gonadotropin analogs and derivatives, and human chorionic gonadotropin agonists and antagonists. In one embodiment, the human chorionic gonadotropin can be synthetic. In another embodiment, the human chorionic gonadotropin can be hCG (see, for example, U.S. Pat. Nos. 6,469,139, 4,400,316, and 4,804,626, incorporated herein by reference). In yet another embodiment, eCG, hCG, and LH are not used in the methods described herein.

In one embodiment, the gonadotropin-releasing hormone can be synthetic. In another embodiment, the gonadotropin-releasing hormone can be in acetate form. In another embodiment, the gonadotropin-releasing hormone can be GnRH (pGlu-His-Trp-Ser-Tyr-Gly-Leu-Arg-Pro-GlyNH₂) (see, for example, U.S. Pat. No. 5,688,506, incorporated herein by reference) or triptorelin (pGlu-His-Trp-Ser-Tyr-D-Trp-Leu-Arg-Pro-Gly-NH₂).

Examples of gonadotropin releasing hormone agonists for use herein include, but are not limited to, leuprolide, nafarelin, buserelin, [DAla⁶, des Gly-NH₂¹⁰]GnRH, [DLys⁶]GnRH, [DAla⁶] GnRH, [2-Me-Ala⁶]GnRH, [D-α-aminobutyroyl⁶, des-GlyNH₂¹⁰]GnRH, triptorelin, lutrelin, goserelin, deslorelin, and histrelin. Generally, gonadotropin-releasing hormone agonists are modeled after the natural gonadotropin releasing hormone decapeptide with specific amino acid substitutions typically at positions 6 and 10. Triptorelin is an example of a gonadotropin releasing hormone agonist with only a single substitution at position 6.

Examples of gonadotropin releasing hormone antagonists include Antide (a decapeptide represented by the formula D-Ac-D-2-Nal¹-DpClPhe²-D-3-Pal³-Ser4-NiLys⁵-D-NicLys⁶-Leu⁷-ILys⁸-Pro⁹-D-Ala¹⁰), [Ac-D4ClDPhe¹, D4ClDPhe², DTrp³, DArg⁶, DAla¹⁰]GnRH, [Ac-4ClDPhe², D₃Pal³, Arg⁵, D₂Nal⁶, DAla¹⁰]GnRH, [Ac-D2-Na¹1, 4ClDPhe², DTrp³, DArg⁶, DAla¹⁰]GnRH, [Ac-D2Nal¹, 4FDPhe², DTrp³, DArg⁶]GnRH, [Ac-D2Nal¹, 4ClDPhe2, DTrp³, DhArg(Et₂)⁶, DAla¹⁰]GnRH, and [Ac—Na¹1, DME4ClPhe², DPal³, Ser⁴, Tyr⁵, DArg⁶, Le⁷, ILys⁸, Pro⁹, DAla¹⁰]GnRH.

In any embodiment described herein, a gonadotropin-releasing hormone of formula (I) can be used

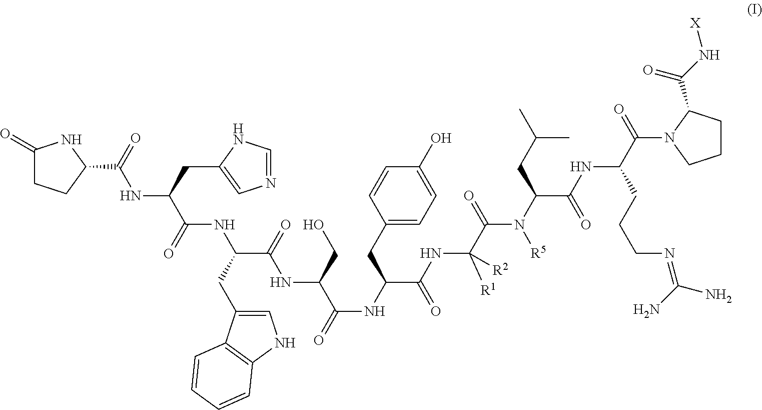

or a solvate, a hydrate, or a pharmaceutically acceptable salt thereof wherein

R¹ and R² are independently in each instance hydrogen, or are independently selected from the group consisting of alkyl, heteroalkyl, cycloalkyl, heterocycloalkyl, haloalkyl, aryl, heteroaryl, arylalkyl, and heteroarylalkyl, each of which is optionally substituted, or R¹ and R² and the attached carbon form a carbocycle or heterocycle;

R⁵ is hydrogen or alkyl; and

X is hydrogen, or X is selected from the group consisting of alkyl, cycloalkyl, heteroalkyl, optionally substituted alkylene-carboxamide, and HNC(O)NR³R⁴, where R³ and R⁴ are in each instance independently selected from the group consisting of hydrogen, alkyl, heteroalkyl and haloalkyl.

In another embodiment, R¹ is a methylene-aryl. In another embodiment, the aryl is phenyl or 4-hydroxyphenyl. In another embodiment, R¹ is a methylene-heteroaryl. In yet another embodiment, the heteroaryl is selected from the group consisting of pyridyl, thiazolyl, pyridazolyl, pyrimidinyl, quinolinyl, pyrazolyl, imidazolyl, pyrrolyl, indolyl, benzopyrazolyl, and benzimidazolyl; and R² is hydrogen or methyl. In various other embodiments, R¹ is 2-methylpropyl, R¹ is 2-naphthylmethyl, R¹ is t-butoxymethyl, R¹ is methyl, R¹ is 4-aminobutyl, R¹ is ethyl, R¹ and R² are methyl, R¹ is 1H-indol-3-yl-methyl, R¹ is 1H-1-benzyl-imidazol-4-yl-methyl, or R¹ is benzyl.

In additional embodiments, R² is hydrogen, R² is hydrogen and the gonadotropin-releasing hormone has the R-configuration at the carbon to which R¹ is attached, R² is hydrogen and the gonadotropin-releasing hormone has the S-configuration at the carbon to which R¹ is attached, or R² is hydrogen and the gonadotropin-releasing hormone is a mixture of gonadotropin-releasing hormones having the R-configuration at the carbon to which R¹ is attached and the S-configuration at the carbon to which R¹ is attached.

In still additional embodiments, X is CH₂(CO)NH₂, X is HN(CO)NH₂, X is ethyl, X is hydrogen, R⁵ is hydrogen, or R⁵ is methyl.

In another embodiment, the gonadotropin-releasing hormone is a hormone of formula I where R¹ is 1H-indol-3-yl-methyl, R² is hydrogen, X is CH₂(CO)NH₂; R⁵ is hydrogen; and the configuration of the carbon to which R¹ is attached is R.

In another embodiment, the gonadotropin-releasing hormone is a hormone of formula I where R¹ is hydrogen, R² is hydrogen, X is CH₂(CO)NH₂; and R⁵ is hydrogen.

In another embodiment, the gonadotropin-releasing hormone is a hormone of formula I where R¹ is 1H-1-benzyl-imidazol-4-yl-methyl, R² is hydrogen, X is ethyl; and R⁵ is hydrogen.

In another embodiment, the gonadotropin-releasing hormone is a hormone of formula I where R¹ is 2-methylpropyl, R² is hydrogen, X is ethyl; and R⁵ is hydrogen.

In yet another embodiment, any one of the previously described embodiments wherein X is CH₂C(O)NH₂ is provided.

In another embodiment, the gonadotropin-releasing hormone is a hormone of formula I where R1 is 2-naphthlymethyl, R2 is hydrogen, X is CH2(CO)NH2; and R5 is hydrogen.

In another embodiment, the gonadotropin-releasing hormone is a hormone of formula I where R1 is t-butoxymethyl, R2 is hydrogen, X is ethyl; R5 is hydrogen; and the configuration of the carbon to which R1 is attached is R.

In another embodiment, the gonadotropin-releasing hormone is a hormone of formula I where R1 is benzyl, R2 is hydrogen, X is CH2(CO)NH2; R5 is hydrogen; and the configuration of the carbon to which R1 is attached is R.

In another embodiment, the gonadotropin-releasing hormone is a hormone of formula I where R1 is t-butoxymethyl, R2 is hydrogen, X is HN(CO)NH2; and R5 is hydrogen.

In another embodiment, the gonadotropin-releasing hormone is a hormone of formula I where R1 is 1H-indol-3-yl-methyl, R2 is hydrogen, X is ethyl; and R5 is hydrogen.

In another embodiment, the gonadotropin-releasing hormone is a hormone of formula I where R1 is methyl, R2 is hydrogen, X is hydrogen; R5 is hydrogen; and the configuration of the carbon to which R1 is attached is R.

In another embodiment, the gonadotropin-releasing hormone is a hormone of formula I where R1 is 1H-indol-3-yl-methyl, R2 is hydrogen, X is ethyl; R5 is methyl; and the configuration of the carbon to which R1 is attached is R.

In another embodiment, the gonadotropin-releasing hormone is a hormone of formula I where R1 is methyl, R2 is hydrogen, X is CH2(CO)NH2; R5 is hydrogen; and the configuration of the carbon to which R1 is attached is R.

The gonadotropin-releasing hormones, such as those described in the formula above, used herein can be administered in the form of pharmaceutically acceptable non-toxic salts or complexes. The salts include acid addition salts such as, for example, hydrochloride, hydrobromide, sulfate, phosphate, nitrate, oxalate, fumarate, gluconate, tannate, maleate, acetate, benzoate, succinate, alginate, malate, ascorbate, tartrate and the like. The complexes can be with metals such as for example zinc, barium, calcium, magnesium, aluminum and the like.

Additional examples of acceptable hormones for use in the methods described herein include, prostaglandins, progestogens, progesterones, androgens, testosterones, estrogens, and estradiols, PMSG, PG-600, derivatives and analogs thereof, combinations thereof, and the like. One or more hormones for synchronizing estrus can be administered. In the methods described herein the hormone used to synchronize estrus can be altrenogest (MATRIX® from Intervet, Inc. Summit, N.J.). The hormone used to synchronize estrus (e.g., altrenogest or any other suitable progestin) can be administered to the gilt by feeding, for example, by mixing the hormone with the gilt's feed or applying the hormone to the gilt's feed. Methods for feeding altrenogest to gilts are well known in the art. For example, in one embodiment, a 0.22% altrenogest solution (or any other suitable concentration) can be used to administer 15 mg of altrenogest per gilt once daily for 14 days in the gilt's feed. In another embodiment, a 0.22% altrenogest solution (or any other suitable concentration) can be used to administer 20 mg of altrenogest per gilt once daily for 18 days in the gilt's feed. Any other suitable regimen for the administration of altrenogest or another hormone for synchronizing estrus (e.g., any other suitable progestin) can be used in accordance with the invention. The hormone for synchronizing estrus can be administered to the gilt by any suitable method known in the art, including by feeding. In one embodiment, the gilt may have had at least one estrus cycle prior to starting administration of the hormone for synchronizing estrus, and that estrus cycle can occur, for example, 4 to 16 days before starting the administration of the hormone for synchronizing estrus.

The amount of the gonadotropin-releasing hormone effective for use in accordance with the methods and compositions described herein depends on many parameters, including the molecular weight of the gonadotropin-releasing hormone, its route of administration, and whether it is in its native form. As in described herein an “effective amount” of the hormone is an amount sufficient to synchronize ovulation or to synchronize the time of insemination in a gilt or a sow using the methods described herein.

The effective amount of the gonadotropin-releasing hormone to be administered to a gilt or a sow can range from about 100 ng to about 2000 μg, about 100 ng to about 1000 μg, about 100 ng to about 500 μg, about 1 μg to about 2000 μg, about 1 μg to about 500 μg, about 1 μg to about 100 μg, about 1 μg to about 50 μg, about 1 μg to about 10 μg, about 10 μg to about 2000 μg, about 10 μg to about 1000 μg, about 10 μg to about 500 μg, about 10 μg to about 100 μg, about 10 μg to about 50 μg, about 50 μg to about 2000 μg, about 50 μg to about 1000 μg, about 50 μg to about 500 μg, about 50 μg to about 300 μg, about 50 μg to about 200 μg, about 100 μg to about 200 μg, about 100 μg to about 300 μg, about 100 μg to about 500 μg, about 100 μg to about 1000 μg, about 200 μg to about 2000 μg, or about 0.05 mg to about 50 mg. In various illustrative aspects, the gonadotropin-releasing hormone can be administered to a gilt or a sow at a dose of about about 1 μg, about 2 μg, about 5 μg, about 10 μg, 20 μg, about 50 μg, about 75 μg, about 100 μg, about 150 μg, about 180 μg, about 200 μg, about 225 μg, about 250 μg, about 300 μg, about 400 μg, about 500 μg, about 750 μg, about 1000 μg, about 1500 μg, or about 2000 μg of the gonadotropin-releasing hormone. The gonadotropin-releasing hormone can be administered in one or more doses. Typically, the gonadotropin-releasing hormone is administered without any additional hormone to synchronize ovulation.

The gonadotropin-releasing hormone in the composition used for the methods described herein can be administered at a concentration of, for example, about 0.1 μg/mL, about 0.5 μg/mL, about 1 μg/mL, about 5 μg/mL, about 10 μg/mL, about 50 μg/mL to about 500 μg/mL, about 50 μg/mL to about 400 μg/mL, about 50 μg/mL to about 300 μg/mL, about 50 μg/mL to about 200 μg/mL, about 50 μg/mL to about 150 μg/mL, about 50 μg/mL to about 250 μg/mL, or about 100 μg/mL. In illustrative embodiments, the composition can be administered in various volumes including for example a dosage volume of 0.1 mL, 0.5 mL, 1 mL, 2 mL, 3 mL, 4mL, or 5 mL. Any suitable volume for administration can be used, depending on, for example, the route of administration to the animal, the size of the animal to which the hormone is being administered, the species of the animal to which the hormone is being administered, and other factors known to those skilled in the art.

In any embodiment described herein, the gonadotropin-releasing hormone is administered in an amount effective to stimulate ovarian follicle ovulation and to synchronize ovulation according to the methods described herein. The dose of the gonadotropin-releasing hormone can be administered using a method selected from the group consisting of 1) use of a deposition catheter, 2) manual administration, 3) injection, or any other art recognized means for administering a pharmaceutical composition, for example, any other art recognized means for vaginally administering a pharmaceutical composition, such as a composition containing a hormone. In one embodiment, the gonadotropin-releasing hormone can be administered to more than one gilt or sow.

Examples of methods for effective gonadotropin-releasing hormone administration, other than vaginal administration, include parenteral administration to the gilt or sow, for example, subcutaneously, intramuscularly, intraperitoneally, intrathecally, or intravenously, or in combination with an acceptable carrier. Suitable means for parenteral administration include needle (including microneedle) injectors, needle-free injectors, and infusion techniques. The parenteral compositions for use in accordance with this invention can be in the form of a reconstitutable lyophilizate comprising one or more doses of the gonadotropin-releasing hormone composition. Examples of parenteral dosage forms include aqueous solutions of the gonadotropin-releasing hormone composition in well-known acceptable liquid carriers such as liquid alcohols, glycols (e.g., polyethylene glycols), glucose solutions (e.g., 5%), esters, amides, sterile water, buffered saline (including buffers like phosphate or acetate; e.g., isotonic saline).

In any embodiment described herein, the gonadotropin-releasing hormone composition for use in the methods described herein can be administered to the gilt or sow locally. Examples of local administration methods for use herein include, topical, intravaginal, and intrarectal. Examples of dosage forms for use in this embodiment include creams, ointments, gels, pastes, powders, lotions, transdermal patches, intrauterine devices, vaginal rings, and vaginal tablets. In one illustrative embodiment, the gonadotropin-releasing hormone composition is administered into the anterior vagina of the gilt or sow. The gonadotropin-releasing hormones may also be formulated in vaginal or rectal compositions such as suppositories, e.g., containing conventional suppository bases such as cocoa butter, carbowaxes, polyethylene glycols or other glycerides, all of which melt at body temperature, yet are solidified at room temperature.

The gonadotropin-releasing hormone may be administered to the gilt or sow by any useful procedures and any effective dose and suitable dosage form can be used, including oral dosage forms known in the art, such as pills, pellets, or capsules, and effective doses can be administered in standard or modified release dosage forms. Modified release dosage formulations include delayed, sustained, pulsed, controlled, targeted, and programmed release formulations.

The gonadotropin-releasing hormone compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

In another illustrative aspect, the gonadotropin-releasing hormone for use in the methods described herein may be in the form of a kit. The kit can comprise a dose or multiple doses of a gonadotropin-releasing hormone as described herein. In this embodiment, the kit can further comprise an applicator for manual administration, a deposition catheter, and/or a syringe for application of the hormone composition to the gilt or sow. In yet another embodiment, the gonadotropin-releasing hormone is in a composition comprising a gel as described herein. In one illustrative embodiment, the kit may comprise the gonadotropin-releasing hormone and the gel separately for mixing before administration to the gilt or sow, or they can be together in one composition. In another embodiment, the kit may comprise the gonadotropin-releasing hormone and the gel admixed in a vessel for immediate administration.

In yet another embodiment, the kit contains instructions for use. The instructions may indicate that the insemination should be through one or more artificial inseminations. The instructions can also provide the timing for administration of the gonadotropin-releasing hormone and the hormone for synchronizing estrus to the gilt as described herein, and the timing for artificial insemination.

In yet another embodiment, an article of manufacture is provided. The article of manufacture can comprise any of the gonadotropin-releasing hormone compositions described herein for use in the methods described herein. The gonadotropin-releasing hormone composition can be in a primary container, for example, a glass vial, such as an amber glass vial with a rubber stopper and/or an aluminum tear-off seal. In another embodiment, the primary container can be plastic or aluminum, and the primary container can be sealed. In another embodiment, the primary container may be contained within a secondary container to further protect the composition from light. The secondary container can be, for example, cardboard. Any of these embodiments also apply to the kit embodiments described above, and any of the gonadotropin-releasing hormone composition embodiments described herein can apply to the article of manufacture.

### EXAMPLE 1

### Study Design and Treatments

All gilts were individually fed MATRIX® as a top-dress for 14 days at the recommended rate of 15 mg/gilt/day. Gilts were withdrawn from MATRIX® feeding (last top feed in the morning on Day 0) and allotted to treatments. Controls were not treated and were inseminated at estrus detected following MATRIX® withdrawal (last top-dress). The remaining gilts received 200 mcg triptorelin, as the acetate, in a 1.2% methylcellulose gel formulation intravaginally 120 hours after MATRIX® withdrawal. OvuGel 1 and OvuGel 2 gilts were administered OvuGel 120 hours after the last feeding of Matrix. OvuGel land OvuGel 2 gilts received a single insemination 24 hours post OvuGel regardless of estrus status. OvuGel 2 gilts that were expressing estrus 24 hours after the first insemination received a second insemination. Control gilts were inseminated as they were detected in estrus and bred each day they remained in estrus.
Morristown North Gilt Trial—Reps 1 through 15
Performance of Gilts—Raw Means

| | | | OvuGel 2 | OvuGel 2 | OvuGel 2 | Treatment, |
|---|---|---|---|---|---|---|
| | Control | OvuGel 1 | (All) | (1 Mating) | (2 Matings) | P< |
| Number of Gilts | 137 | 134 | 134 | 67 | 67 | — |
| HNS to 1^{st} Matrix, d | 7.5 | 7.5 | 7.6 | 7.3 | 7.9 | 0.97 |
| Last Matrix to Estrus, d | 6.9 | 6.4 | 6.7 | 7.7 | 6.4 | 0.01 |
| In Estrus at 1^{st} Mating, % | 100 | 41.0 | 38.8 | 13.4 | 64.2 | 0.01 |
| No Estrus Detected, % | 12.4 | 41.0 | 35.8 | 71.6 | 0 | 0.01 |
| Number of Doses | 2.0 | 1.0 | 1.5 | 1.0 | 2.0 | 0.01 |
| Returns, % | 5.1 | 14.2 | 12.8 | 22.7 | 3.0 | 0.01 |
| Pregnant, % | 81.0^{a} | 76.9 | 80.3 | 63.6 | 97.0 | 0.01 |

^{a}In addition to the gilts bred by day 7 post-Matrix, this calculation includes 19 gilts (16 pregnant) bred on day 8, 4 gilts (3 pregnant) bred on day 9, 1 pregnant gilt bred on day 10, and 1 pregnant gilt bred on day 13 post-Matrix as well as 17 gilts that never expressed estrus and were counted as not pregnant.

Expression of Estrus Post-MATRIX

| | Control | OvuGel 1 | OvuGel 2 |
|---|---|---|---|
| Number of Gilts | 137 | 134 | 134 |
| 1^{st} Expression of Estrus on d 5, % | 1.5 | — | — |
| 1^{st} Expression of Estrus on d 6, % | 29.9 | 41.0 | 38.8 |
| 1^{st} Expression of Estrus on d 7, % | 38.0 | 13.4 | 17.9 |
| 1^{st} Expression of Estrus on d 8, % | 13.9 | 3.0 | 1.5 |
| 1^{st} Expression of Estrus on d 9, % | 2.9 | 1.5 | 3.0 |
| 1^{st} Expression of Estrus on d 10, % | 0.7 | — | 3.0 |
| Percent not expressing Estrus | 12.4 | 41.0 | 35.8 |

Pregnancy Rate Based on When Gilts Expressed Estrus

| | Control | OvuGel 1 | OvuGel 2 |
|---|---|---|---|
| Number of Gilts | 137 | 134 | 134 |
| Pregnant (%) - d 5 Estrus | 100 | — | — |
| Pregnant (%) - d 6 Estrus | 95.1 | 83.6 | 92.3 |
| Pregnant (%) - d 7 Estrus | 94.2 | 77.8 | 95.7 |
| Pregnant (%) - d 8 Estrus | 84.2 | 0 | 0 |
| Pregnant (%) - d 9 Estrus | 75.0 | 50.0 | 0 |
| Pregnant (%) - d 10 Estrus | 100 | — | 50.0 |
| Pregnant (%) - No Estrus | 0 | 76.4 | 70.8 |
| Pregnant (%) - Total | 80.3 | 76.9 | 80.3 |

Morristown South Gilt Trial—Reps 1 through 15
Performance of Gilts—Raw Means

| | | | OvuGel 2 | OvuGel 2 | OvuGel 2 | Treatment, |
|---|---|---|---|---|---|---|
| | Control | OvuGel 1 | (All) | (1 Mating) | (2 Matings) | P< |
| Number of Gilts | 134 | 128 | 128 | 31 | 97 | — |
| HNS to 1^{st} Matrix, d | 8.3 | 8.3 | 8.3 | 7.8 | 8.5 | 0.29 |
| Last Matrix to Estrus, d | 6.4 | 6.2 | 6.1 | 6.4 | 6.1 | 0.01 |
| In Estrus at 1^{st} Mating, % | 98.5 | 67.2 | 73.4 | 38.7 | 84.5 | 0.01 |
| No Estrus Detected, % | 3.0 | 16.4 | 14.1 | 51.6 | 2.1 | 0.01 |
| Number of Doses | 2.0 | 1.0 | 1.8 | 1.0 | 2.0 | 0.01 |
| Returns, % | 0.8 | 13.4 | 5.6 | 13.8 | 3.1 | 0.01 |
| Pregnant, % | 93.9^{a} | 82.5 | 93.7 | 82.8 | 96.9 | 0.01 |

^{a}In addition to the gilts bred by day 7 post-Matrix, this calculation includes 4 pregnant gilts bred on day 8, 3 pregnant gilts bred on day 10 and 1 pregnant gilt bred on day 13 post-Matrix as well as 4 gilts that never expressed estrus and were counted as not pregnant.

Expression of Estrus Post-MATRIX

| | Control | OvuGel 1 | OvuGel 2 |
|---|---|---|---|
| Number of Gilts | 134 | 128 | 128 |
| 1^{st} Expression of Estrus on d 5, % | 5.2 | 2.3 | 4.7 |
| 1^{st} Expression of Estrus on d 6, % | 60.4 | 65.6 | 68.8 |
| 1^{st} Expression of Estrus on d 7, % | 25.4 | 15.6 | 9.4 |
| 1^{st} Expression of Estrus on d 8, % | 3.0 | 1.6 | 3.1 |
| 1^{st} Expression of Estrus on d 9, % | — | — | — |
| 1^{st} Expression of Estrus on d 10, % | 2.2 | — | — |
| 1^{st} Expression of Estrus on d 13, % | 0.7 | — | — |
| Percent not expressing Estrus | 3.0 | 14.8 | 14.1 |

Pregnancy Rate Based on When Gilts Expressed Estrus

| | Control | OvuGel 1 | OvuGel 2 |
|---|---|---|---|
| Number of Gilts | 134 | 128 | 128 |
| Pregnant (%) - d 5 Estrus | 100 | 100 | 100 |
| Pregnant (%) - d 6 Estrus | 96.2 | 86.7 | 97.7 |
| Pregnant (%) - d 7 Estrus | 97.0 | 73.7 | 100 |
| Pregnant (%) - d 8 Estrus | 100 | 0 | 25.0 |
| Pregnant (%) - d 9 Estrus | — | — | — |
| Pregnant (%) - d 10 Estrus | 100 | — | — |
| Pregnant (%) - d 13 Estrus | 100 | — | — |
| Pregnant (%) - No Estrus | 0 | 78.9 | 82.4 |
| Pregnant (%) - Total | 93.9 | 82.5 | 93.7 |

Brentwood Gilt Trial—Reps 1 through 15
Performance of Gilts—Raw Means

| | | | OvuGel 2 | OvuGel 2 | OvuGel 2 | Treatment, |
|---|---|---|---|---|---|---|
| | Control | OvuGel 1 | (All) | (1 Mating) | (2 Matings) | P< |
| Number of Gilts | 148 | 148 | 145 | 85 | 60 | — |
| HNS to 1^{st} Matrix, d | 10.1 | 10.2 | 10.2 | 9.9 | 10.6 | 0.06 |
| Last Matrix to Estrus, d | 7.3 | 7.2 | 7.0 | 7.6 | 6.4 | 0.01 |
| In Estrus at 1^{st} Mating, % | 100 | 38.5 | 38.6 | 23.5 | 60.0 | 0.01 |
| No Estrus Detected, % | 10.8 | 21.6 | 22.1 | 37.6 | 0 | 0.01 |
| Number of Doses | 1.9 | 1.0 | 1.4 | 1.0 | 2.0 | 0.01 |
| Returns, % | 1.4 | 8.1 | 8.3 | 12.9 | 1.7 | 0.01 |
| Pregnant, % | 78.4 ^{a} | 83.8 | 84.8 | 76.5 | 96.7 | 0.03 |

^{a} In addition to the gilts bred by day 7 post-Matrix, this calculation includes 39 gilts (35 of which were pregnant) bred on days 8-14 post-Matrix as well as 11 gilts that were bred on days 15-30 post-Matrix and 16 gilts that never expressed estrus. These 27 gilts that expressed estrus after day 14 or not at all, were recorded as not pregnant.

Expression of Estrus Post-MATRIX

| | Control | OvuGel 1 | OvuGel 2 |
|---|---|---|---|
| Number of Gilts | 148 | 148 | 145 |
| 1^{st} Expression of Estrus on d 5, % | 2.7 | 2.7 | 1.4 |
| 1^{st} Expression of Estrus on d 6, % | 28.4 | 35.8 | 37.2 |
| 1^{st} Expression of Estrus on d 7, % | 24.3 | 14.2 | 19.3 |
| 1^{st} Expression of Estrus on d 8, % | 13.5 | 7.4 | 7.6 |
| 1^{st} Expression of Estrus on d 9, % | 6.1 | 10.8 | 9.0 |
| 1^{st} Expression of Estrus on d 10, % | 1.4 | 6.1 | 2.8 |
| 1^{st} Expression of Estrus on d 11-14, % | 5.4 | 1.4 | 0.7 |
| Percent not expressing Estrus | 18.2 | 21.6 | 22.1 |

Pregnancy Rate Based on When Gilts Expressed Estrus

| | Control | OvuGel 1 | OvuGel 2 |
|---|---|---|---|
| Number of Gilts | 148 | 148 | 145 |
| Pregnant (%) - d 5 Estrus | 100 | 100 | 100 |
| Pregnant (%) - d 6 Estrus | 97.6 | 94.3 | 88.9 |
| Pregnant (%) - d 7 Estrus | 100 | 66.7 | 92.9 |
| Pregnant (%) - d 8 Estrus | 95.0 | 81.8 | 54.5 |
| Pregnant (%) - d 9 Estrus | 88.9 | 93.8 | 76.9 |
| Pregnant (%) - d 10 Estrus | 100 | 88.9 | 100 |
| Pregnant (%) - d 11-14 Estrus | 75.0 | 100 | 100 |
| Pregnant (%) - No Estrus | 0 | 68.8 | 81.3 |
| Pregnant (%) - Total | 78.4 | 83.8 | 84.8 |

### EXAMPLE 2

### Test Substance

Triptorelin (pGlu-His-Trp-Ser-Tyr-D-Trp-Leu-Arg-Pro-Gly-NH₂) was supplied in the acetate form, from Bachem, Torrance, Calif. (Item H-4075 CGMP grade). Triptorelin gel (200 μg/2 mL) was formulated by Argenta (Auckland, NZ) in a gel composed of Methocel Premium A4000 (Dow Chemical), citrate buffer (pH 5.5), NaCl, methionine and EDTA (as potential stabilizers) and methyl and propyl parabens (as preservatives). Fifty-four milliliters of triptorelin gel (100 mcg triptorelin acetate/mL) was packaged in Amber Borosilicate Glass Serum Vials (610206-50) with a Gray Butyl Pharmaceutical Serum Vial Stopper (73828A-SS) with a Standard Aluminum Seal (SAS2ONAT). The triptorelin gel vehicle contained the same formulation excipients as in triptorelin gel, except it did not contain triptorelin or the potential stabilizers, methionine and EDTA. MATRIX® was supplied as the US commercially available form.

### EXAMPLE 3

### Estrus Observation

For post-treatment estrus detection, gilts were housed in individual pens. Boars were housed in separate rooms, and/or at least 12 m away and downwind. To elicit signs of estrus, a mature boar was walked slowly in the alley in front of the gilts' crates, exposing each test gilt to visual, auditory and olfactory signals from the boar for up to 5 minutes. In keeping with standard practice at commercial farms, while the boar was near the front of the gilt's crate, estrus was tested by an experienced person applying back pressure to the midsection of the gilt combined with side rubbing. Estrus was confirmed when a gilt stood rigidly to the back pressure, with no vocalization and with some indication of an ear reflex. Estrus detection was performed on gilts once daily from Day 0 to Day 7, as appropriate for the embodiment.

### EXAMPLE 4

### Administration

A single 2 mL dose of triptorelin gel or vehicle gel was deposited within approximately 1-2 cm posterior to the cervix with a catheter similar to those used for artificial insemination. The dose was delivered using a standard multi-dose applicator attached to the catheter. A new disposable sheath, which surrounds the catheter, was used for each gilt.

### EXAMPLE 5

### Statistical Analysis

Data were analyzed using the Mixed Models procedures of SAS. Models included main effects of time of treatment following the last feeding of MATRIX®, replicate, and treatment by replicate interaction. Differences between treatments were tested on least squares means estimates using the T test at P<0.05. Data for analysis were tested for assumptions of data normality.

### EXAMPLE 6

### Preparation of Triptorelin-Containing Composition

Methylparaben sodium salt and propylparaben sodium salt were added to purified water with mixing and mixing continued for 5-10 minutes. Sodium chloride USP was then added with mixing for another 10-15 minutes followed by the addition of L-methionine with mixing for 10-15 minutes. Sodium citrate USP was then also added with mixing for another 10-20 minutes.

In a separate mixer, citric acid was added to purified water and was mixed 5-10 minutes before the addition of triptorelin acetate, and mixing then continued for 10-20 minutes. The paraben-containing composition was then added to the triptorelin-containing composition and mixed for 10-15 minutes. Methylcellulose was then slowly added to avoid clumping and mixing continued for another 30-60 minutes. The pH of the mixture was then checked and citric acid in purified water was added as necessary to adjust the pH of the composition.

### EXAMPLE 7

### Example Formulations

Example formulations for the composition described in this application are shown in Tables 33 and 34.

TABLE 33

| | | Weight (% |
|---|---|---|
| Ingredient | Function | w/v) |
| Methylparaben, sodium | Anti-microbial | 0.0900 |
| salt (USNF) | preservative | |
| Propylparaben, sodium | Anti-microbial | 0.0100 |
| salt (USNF) | preservative | |
| Sodium chloride, laboratory | Tonicity agent | 0.910 |
| reagent | | |
| Sodium citrate, dihydrate | Buffering agent | 0.186 |
| L-Methionine, laboratory | Stabilizing agent | 0.100 |
| reagent | | |
| Citric acid, anhydrous | Buffer | 0.0700 |
| Triptorelin acetate | Active Pharmaceutical | 0.0100 |
| | Ingredient (API) | |
| Water (USNF) | Dissolving solvent | 98.4 |
| Methylcellulose (A4M Premium) | Thickening agent | 1.20 |
| (USP) | | |

TABLE 34

| | | | Amount per |
|---|---|---|---|
| | Quality | | 100 mg |
| Component | Standard | Function | % w/w |
| Triptorelin Acetate | In house | Drug Substance | 11.0 mg |
| | | | 0.011%* |
| Purified Water | USP | Solvent | 97.6 g |
| | | | 97.54%* |
| Methylparaben, | NF | Preservative | 89.0 mg |
| Sodium Salt** | | | 0.089%* |
| Propylparaben, | NF | Preservative | 10.0 mg |
| Sodium Salt** | | | 0.010* |
| Sodium Chloride | USP | Tonicity agent | 901 mg |
| | | | 0.901% |
| L-Methionine | USP | Stabilizing agent | 99.0 mg |
| | | | 0.099% |
| Sodium Citrate | USP | Buffering agent | 184 mg |
| | | | 0.184% |
| Citric Acid | USP | Buffering agent | 69.0 mg |
| | | | 0.069%* |
| Methycellulose | USP | Viscosity modifier | 1.1 g |
| | | | 1.10%* |

*Nominal amount
**Tested to compendial standard

1. A method for synchronizing time of insemination in a gilt, the method comprising the steps of:
administering to the gilt a hormone for synchronizing estrus;
administering to the gilt a single dose of a gonadotropin-releasing hormone for synchronizing ovulation, without administration of any other hormone for synchronizing ovulation, wherein the gonadotropin-releasing hormone is administered on the fifth day after the last daily administration of the hormone for synchronizing estrus;
inseminating the gilt, without monitoring estrus, on the sixth day after the last daily administration of the hormone for synchronizing estrus;
monitoring estrus on the seventh day after the last daily administration of the hormone for synchronizing estrus, and
i) if the gilt is in estrus on the seventh day after the last daily administration of the hormone for synchronizing estrus, inseminating the gilt on the seventh day after the last daily administration of the hormone for synchronizing estrus, or ii) if the gilt is not in estrus on the seventh day after the last daily administration of the hormone for synchronizing estrus, not inseminating the gilt on the seventh day after the last daily administration of the hormone for synchronizing estrus.

2. The method according to claim 1 wherein the gonadotropin releasing hormone has the formula 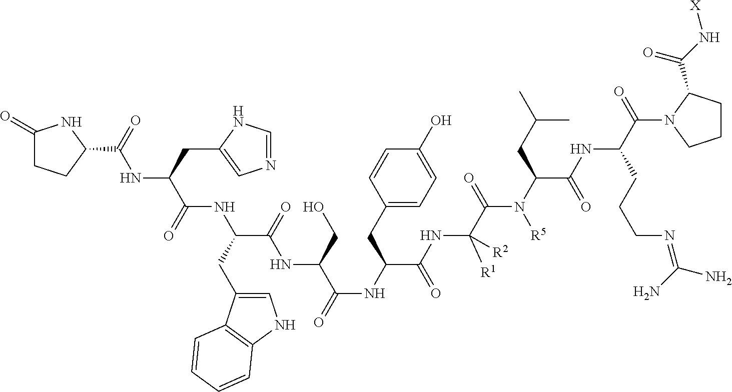
or a solvate, a hydrate, or a pharmaceutically acceptable salt thereof wherein
R¹ and R² are independently in each instance hydrogen, or are independently selected from the group consisting of alkyl, heteroalkyl, cycloalkyl, heterocycloalkyl, haloalkyl, aryl, heteroaryl, arylalkyl, and heteroarylalkyl, each of which is optionally substituted, or R¹ and R² and the attached carbon form a carbocycle or heterocycle;
R⁵ is hydrogen or alkyl; and
X is hydrogen, or X is selected from the group consisting of alkyl, cycloalkyl, heteroalkyl, optionally substituted alkylene-carboxamide, and HNC(O)NR³R⁴, where R³ and R⁴ are in each instance independently selected from the group consisting of hydrogen, alkyl, heteroalkyl and haloalkyl.

3. The method according to claim 2 wherein the gonadotropin-releasing hormone is selected from the group consisting of compounds of the formula of claim 2 wherein
a) R¹ is 1H-indol-3-yl-methyl, R² is hydrogen, X is CH₂(CO)NH₂; R⁵ is hydrogen; and the configuration of the carbon to which R¹ is attached is R;
b) R¹ is hydrogen, R² is hydrogen, X is CH₂(CO)NH₂; and R⁵ is hydrogen;
c) R¹ is 1H-1-benzyl-imidazol-4-yl-methyl, R² is hydrogen, X is ethyl; and R⁵ is hydrogen;
d) R¹ is 2-methylpropyl, R² is hydrogen, X is ethyl; and R⁵ is hydrogen;
e) R¹ is 2-naphthlymethyl, R² is hydrogen, X is CH₂(CO)NH₂; and R⁵ is hydrogen;
f) R¹ is t-butoxymethyl, R² is hydrogen, X is ethyl; R⁵ is hydrogen; and the configuration of the carbon to which R¹ is attached is R;
g) R¹ is benzyl, R² is hydrogen, X is CH₂(CO)NH₂; R⁵ is hydrogen; and the configuration of the carbon to which R¹ is attached is R;
h) R¹ is t-butoxymethyl, R² is hydrogen, X is HN(CO)NH₂; and R⁵ is hydrogen;
i) R¹ is 1H-indol-3-yl-methyl, R² is hydrogen, X is ethyl; and R⁵ is hydrogen;
j) R¹ is methyl, R² is hydrogen, X is hydrogen; R⁵ is hydrogen; and the configuration of the carbon to which R¹ is attached is R;
k) R¹ is 1H-indol-3-yl-methyl, R² is hydrogen, X is ethyl; R⁵ is methyl; and the configuration of the carbon to which R¹ is attached is R;
l) R¹ is methyl, R² is hydrogen, X is CH₂(CO)NH₂; R⁵ is hydrogen; and the configuration of the carbon to which R¹ is attached is R;
m) R¹ is 4-aminobutyl, R² is hydrogen, X is HN(CO)NH₂; R⁵ is hydrogen; and the configuration of the carbon to which R¹ is attached is R;
n) R¹ is methyl, R² is methyl, X is HN(CO)NH₂; and R⁵ is hydrogen; and
o) R¹ is ethyl, R² is hydrogen, X is hydrogen; R⁵ is hydrogen; and the configuration of the carbon to which R¹ is attached is R.

4. The method according to claim 1 wherein the insemination is an artificial insemination.

5. (canceled)

6. The method according to claim 1 wherein the gonadotropin-releasing hormone is administered in an effective amount and the effective amount of the gonadotropin-releasing hormone is about 100 μg to about 300 μg.

7. The method according to claim 1 wherein the gonadotropin-releasing hormone is administered in an effective amount and the effective amount of the gonadotropin-releasing hormone is about 200 μg.

8.-9. (canceled)

10. The method of claim 1 wherein the gonadotropin-releasing hormone is at a concentration of about 100 μg/mL.

11. The method according to claim 1 wherein the dose of the gonadotropin-releasing hormone is administered using a method selected from the group consisting of use of a deposition catheter, manual administration, and injection.

12. The method of claim 11 wherein the gonadotropin-releasing hormone is administered using a deposition catheter.

13.-16. (canceled)

17. The method according to claim 1 wherein the gonadotropin-releasing hormone is administered in a composition comprising methylcellulose.

18. The method of claim 17 wherein the composition comprises about 0.5 weight % to about 4.0 weight % of methylcellulose.

19.-20. (canceled)

21. The method of claim 1 wherein the gonadotropin-releasing hormone is administered intravaginally.

22. The method of claim 21 wherein the gonadotropin-releasing hormone is administered into the anterior vagina.

23.-24. (canceled)

25. The method of claim 1 wherein the gonadotropin-releasing hormone is triptorelin acetate.

26. The method of claim 1 wherein the hormone that synchronizes estrus is altrenogest.

27. The method according to claim 1 wherein the gonadotropin-releasing hormone is in a composition and the composition further comprises a stabilizer wherein the stabilizer is L-methionine.

28. The method of claim 1 wherein the gonadotropin-releasing hormone is in a composition with a pH of about 5 to about 6.

29. The method of claim 1 wherein the gonadotropin-releasing hormone is in a composition further comprising a preservative and wherein the preservative is selected from the group consisting of methylparaben and propylparaben.

30.-31. (canceled)

32. The method of claim 1 wherein the composition comprises methylparaben in an amount of about 0.09% weight per volume, propylparaben in an amount of about 0.01% weight per volume, sodium chloride in an amount of about 0.91% weight per volume, sodium citrate in an amount of about 0.186% weight per volume, L-methionine in an amount of about 0.1% weight per volume, citric acid in an amount of about 0.07% weight per volume, triptorelin in an amount of about 0.01% weight per volume, and methycellulose in an amount that provides a viscosity of about 250 cP to about 400 cP.

33. The method according to claim 1 wherein the gonadotropin-releasing hormone is in an excipient selected from the group consisting of buffered saline, a liquid alcohol, a glycol, a glucose solution, an ester, an amide, and sterile water.

34. The method of claim 33 wherein the excipient further comprises a pH buffering agent selected from the group consisting of an acetate buffer, a borate buffer, a carbonate buffer, a citrate buffer, a phosphate buffer, hydrochloric acid, sodium hydroxide, magnesium oxide, monopotassium phosphate, bicarbonate, ammonia, carbonic acid, sodium citrate, citric acid, acetic acid, and disodium hydrogen phosphate.

35. The method of claim 1 further comprising the step of exposing the gilt to a boar.

36. The method of claim 1 wherein the hormone for synchronizing estrus is administered by feeding.

37. The method of claim 1 wherein the gonadotropin-releasing hormone is administered about 118 to about 124 hours after the last daily administration of the hormone for synchronizing estrus.

38. The method of claim 1 wherein the gonadotropin-releasing hormone is administered about 124 to about 132 hours after the last daily administration of the hormone for synchronizing estrus.

39. The method of claim 1 wherein the gilt is inseminated on the sixth day after the last daily administration of the hormone for synchronizing estrus at about 15 to about 32 hours after administration of the gonadotropin-releasing hormone.

40. The method of claim 1 wherein the gilt had at least one estrus cycle prior to starting administration of the hormone for synchronizing estrus.
